(19) United States
(12) Patent Application Publication
(10) Pub. No.: US 2021/0153920 A1
(43) Pub. Date: May 27, 2021

(54) APPARATUS AND METHOD FOR ABLATION OF SOFT TISSUE SURROUNDING A BREAST CAVITY FOLLOWING LUMPECTOMY

(71) Applicant: SenoGuard, Inc., Seal Beach, CA (US)

(72) Inventors: Greig Eric Altieri, Charlotte, NC (US); Diana Lee Tucker, Seal Beach, CA (US); Roberta Lee, Seal Beach, CA (US); Darius S. Francescatti, Barrington, IL (US)

(73) Assignee: SenoGuard, Inc., Seal Beach, CA (US)

(21) Appl. No.: 17/102,139

(22) Filed: Nov. 23, 2020

Related U.S. Application Data
(60) Provisional application No. 62/939,198, filed on Nov. 22, 2019.

(51) Int. Cl. A61B 18/02

(52) U.S. Cl. CPC A61B 18/02; A61B 2018/0022; A61B 2018/0262

(57) ABSTRACT

Methods and systems for delivering a cryogenic fluid to an inner surface of a surgical excision cavity, such as following breast tumor removal, are described. A single or dual balloon configuration can be utilized to deliver a cryogenic fluid to necrose tissue surrounding the tumor cavity in order to reduce the risk of recurrence of the cancer. The balloon can be optimally configured for maximum contact with the breast tumor bed.

### CROSS-REFERENCE TO RELATED APPLICATION

This application claims the benefit of U.S. Provisional Application No. 62/939,198 (Attorney Docket No. 57648-703.101, filed Nov. 22, 2019, the entire content of which is incorporated herein by reference.

### BACKGROUND OF THE INVENTION

### 1. Field of the Invention

The present invention relates generally to medical devices and methods and more particularly to methods and device for treating a margin of tissue adjacent to a surgical cavity in a breast following the removal of a cancerous tumor.

The current standard of care for breast-conserving surgery is irradiation of the whole breast for patients in a high risk category or a partial resection of the breast containing the cancer in order to eradicate residual undiscoverable nidus of microscopic cancer following tumor excision. Such radiation therapy, however, can have serious side effects for the patient. Short term complications include skin burns, induration of the breast in the operative area, commensurate tissue distortion and a poor cosmetic result. Long term complications include lymphedema (swelling) of the arm, delayed cardio-toxicity, and (rarely) an occurrence of a secondary non-breast cancer.

Newer technologies provide more targeted approaches to both intra-operative or post-operative adjuvant therapy effecting a resultant reduction in risk of future breast cancer recurrence. Targeted adjuvant technologies are administered to eradicate the surrounding perimeter of breast tissue that may harbor microscopic nests of cancerous tissue in the surrounding 5 to 20 mm rim of breast tissue created by the excision. For instance, intraoperative radiotherapy can be delivered in conjunction with breast conserving surgery (partial mastectomy/lumpectomy). Such radiotherapy may be delivered in the operating room as part of an operative procedure through an operative incision.

While such newer technologies are both convenient and clinically effective for the patient, they still suffer from the risks associated with radiation exposure. Moreover, the delivery of radiation is expensive in both time and cost to the healthcare system.

Other ablative technologies are in development for the post-excision marginal tissue treatment of breast tumor cavities including radio-frequency ablation, high frequency ultrasound, microwave laser therapy to treat the tissue surrounding the surgical cavity. Cryoablation has been proposed for other body cavity tissue ablation procedures, such as endometrial uterine ablation and intracardiac ablation for treating arrhythmias. Cryoablation has been found to generate a greater and more diverse antigenic residue the protective immune response and providing a diversity of immune targets for pharmacological treatment.

### 2. Listing of the Background Art

Cryogenic energy has been used for years for ablation of soft tissue. In the form of a cryogenic balloon the majority of previous devices have been aimed at the treatment of tissues along the walls of a vessel or lumen (U.S. Pat. Nos. 9,414,878 and 9,402,676), at the junction of a cavity and a vessel or lumen (U.S. Pat. No. 7,740,627), or within a specific and complicated body cavity (U.S. Pat. No. 9,603,650). Double-walled safety balloons for cryogenic treatment of the vasculature are described in U.S. Pat. No. 6,811,550. Double-walled cryogenic balloons for performing pulmonary vein ablation for the treatment of atrial fibrillation are described in U.S. Pat. No. 9,033,965. Cryogenic systems for treating a uterus are available from Channel Medsystems, Inc., San Francisco, Calif., USA, and described in U.S. Patent Application 2012/0197245.

### SUMMARY OF THE INVENTION

A balloon system for supplying cryogenic energy to a surgical cavity is described herein. The balloon is inserted into the surgical cavity through a previously created surgical incision site and/or through an additional percutaneous tract. Following placement, a selected cryogenic agent is administered through a channel into the balloon which expands the balloon until circumferential contact is made with the walls of the tumor cavity. Because the balloon system is not placed within a vessel or lumen, no provision for a guidewire system is necessary. Preferably the balloon is optimally expanded or configured for maximum contact with the walls of the surgical cavity.

The balloon system is coupled to a cryogenic fluid delivery system with a distal outflow port and one or more inflow port(s) for supplying cooling energy to the interior of the balloon and removing the latent heat of the surrounding tissue inducing necrosis of the surrounding targeted rim of tissue. The cryogenic fluid delivery system may be as simple as a handheld fluid delivery device or as complicated as a microprocessor controlled closed loop fluid delivery system depending on the selected cryogenic fluid.

Another embodiment of the balloon system for supplying cryogenic energy to the breast tumor cavity has a secondary fluid path with a separate distal outflow port and one or more inflow port(s) for air, CO₂ gas, saline, or other fluid media or gas used to expand the balloon within the surgical cavity and act as a heat sink to reduce the targeted tissue temperature before the delivery of the selected cryogenic fluid thus inducing a cryoablative temperature with subsequent necrosis of the targeted tissue surrounding the surgical cavity. Pre-expansion of the balloon within the surgical cavity facilitates the optimal extraction of latent energy from the surrounding targeted tissue. The reduction of latent energy (tissue heat) from targeted tissue facilitates and enhances the precise delivery of cryoablative tissue necrosis to the targeted perimeter rim of breast tissue. Added benefit will include an overall reduction in total operative time (anesthesia and surgical).

Another embodiment of the balloon system includes a secondary balloon within the tissue contacting balloon supplied by a secondary fluid such as air, CO₂ gas, saline, or other fluid media or gas. This balloon will have a distal outflow port and one or more inflow port(s) that can be used to expand the tissue contacting balloon (within the tumor cavity) and act as a heat sink to reduce normal tissue temperatures prior to delivery of the cryogenic fluid. As noted in the preceding embodiment, pre-expansion of the balloon within the cavity facilitates the most efficient use of the cryogenic fluid in extracting the maximum amount of latent energy from the surrounding targeted tissue thus leading to optimal penetration of cryoablative necrosis to the targeted perimeter breast tissue with resultant decreased procedure times.

Another embodiment of the balloon system for supplying cryogenic energy to the targeted breast tumor cavity perimeter incorporates a suction path exterior to the tissue contacting balloon element. It provides a suction pathway to the interface between balloon surface and the tissue cavity wall. This suction path can be added to any of the balloon configurations described and can/will be used to eliminate any gap, whether air or fluid, interposed outer surface of the tissue contacting balloon and targeted tissue thus assuring precise geographic delivery of cryogenic energy to the targeted tissue.

The balloons will usually be non-distensible with an elasticity below 10%, usually below 5%, when inflated to the operating pressures herein, typically from 100 kPa to 500 kPa. Suitable balloon materials include but are not limited to polyethylene terephthalate (PET), polyamides, nylons, engineered nylons (such as Pebax®, Grilamid®, Vestamid®), and the like.

In specific instances, the present invention provides methods for adjuvant treatment of the tissue surrounding the surgical cavity after removal of a cancerous tumor. Adjuvant therapy is administered to a circumferential rim of tissue surrounding the excisional cavity to ensure that all cancerous cells have been destroyed. The 5 mm to 15 mm circumferential perimeter surrounding the surgical cavity may potentially contain residual rests of undetectable cancer. By creating a zone of cryoablative temperatures (cellular destructive temperatures) surrounding the surgical cavity to a defined circumferential distance, all cellular content, normal and cancerous, is destroyed (killed or necrosed) leaving a residue of nuclear and cellular antigenic material for use by the innate immune system to protect against recurrence and/or lead to the development of new therapeutic remedies.

In another embodiment, a post-surgical method for treating a defined perimeter surrounding the surgical cavity following the excision of a breast cancer is described. This embodiment describes a cryoenergy delivery system comprising an inflatable balloon configured probe delivering cryogenic fluid capable of achieving cryogenic cellular and tissue destructive temperatures to targeted depths. The apparatus is configured to be inserted through a per-cutaneous incision in the post-operative period after definitive post-operative pathological examination of the surgical specimen. The probe device will contain a drainage system to evacuate fluid from the surgical cavity prior to and (if needed) during the delivery of cryotherapy to the targeted circumference of the cavity. The probe balloon will be inflatable using cryogenic fluid. Inflation of the balloon will assure contact with 100% of the cavity wall surface during delivery of the treatment. The entire procedure will be monitored during delivery with real time intra operative imaging means. Cryo-ablative temperatures will be monitored to assure that lethal temperatures are delivered to the entire targeted peri-cavity rim and to the predetermined depth of penetration. Cooling is preferably carried out via a liquid cryogen to and from the targeted post-surgical cavity. Alternatively, the cryofluid may undergo a phase change from liquid to gas and undergo enthalpic heat absorption. Another embodiment will employ a gas that expands while undergoing Joule Thomson heat absorption.

In another embodiment, the cryoenergy delivery structure comprises a balloon where an expandable inner structure can be deployed within the treating balloon while the cryogenic fluid is circulated within the expanded balloon. Expansion may be carried out by manipulation of a pull wire that expands a multifilament segment for better contact with the targeted tissue. In one embodiment, the structure is radially expanded.

In another embodiment, the cryoenergy delivery structure comprises a double-walled balloon wherein the inner balloon is expanded with a secondary (typically non-cooled) fluid and the cryogenic fluid is circulated between the outer balloon and the inner expanded balloon.

In another embodiment the cryogenic fluid undergoes a Jules-Thomson expansion to extract energy from the tissue of the walls comprising the post-surgical cavity following the removal of a cancerous tumor.

In another embodiment the cryogenic fluid uses transition fluid (i.e., neither liquid, nor gas) to extract energy from the tissue of the walls comprising the post-surgical cavity following the removal of a cancerous tumor.

In another embodiment the rate of thawing after the freeze is controlled by warming of the cryogen. This step of thawing may require warming of the cryogen to 43° C.

In another embodiment, cryoablative energy is applied to the post-surgical cavity following removal of the cancer in order induce a cytotoxic temperature at or below −40° C., usually in a range from −40° C. to −200° C. in tissue to the depths previously recited herein.

In another embodiment, cytotherapeutic cycles are delivered (below −40° C.) followed by a period of warming after which another cyrotherapeutic freeze cycle is repeated, once, twice, or more. Temperatures are monitored throughout all treatment cycles. At least two freeze cycle are delivered with an achieved or measured temperature of −40° C. or less to the designated peripheral boundary of the treatment area, typically to a depth of 5 mm to 15 mm deep, often to a depth of 5 mm to 20 mm. The freeze area temperature gradient is actively measured during the procedure for freeze temperature achieved, distance from the balloon surface, time duration of freeze and the geographical extent of therapeutic freeze temperature distance from the balloon surface. Temperature measurement and other parameters are monitored and recorded from start of initial freeze to completion of therapy.

### BRIEF DESCRIPTION OF THE DRAWINGS

FIGS. 1A-1E illustrate in cross-section a breast and the steps for treatment of a breast cancer using a cryogenic balloon.

FIG. 2 illustrates in a cross-sectional view a single balloon system for use with a cryogenic fluid that provides the delivery of a cryogenic fluid to the surgical cavity within the breast.

FIG. 3 illustrates in a cross-sectional view a single balloon system for use with a cryogenic fluid that provides the delivery of a cryogenic fluid to the surgical cavity within the breast and provides a secondary fluid path for balloon expansion and preliminary heat removal.

FIG. 4 illustrates in cross-sectional view a dual balloon system for use with a cryogenic fluid that provides the delivery of a cooling agent to the surgical cavity within the breast.

FIG. 5 illustrates in a cross-sectional view a single balloon system for use with a cryogenic fluid that provides the delivery of a cryogenic fluid to the surgical cavity within the breast and provides a suction path exterior to the tissue contacting balloon.

### DETAILED DESCRIPTION

Cryo-balloon systems are configured to deliver cryogenic temperatures. e.g. freezing temperatures below −40° C., to the surrounding breast tissue perimeter defined by the walls of a surgical cavity post excision of a breast cancer to a distance of from 5 mm to 15 mm as defined by standard adjuvant therapeutic breast cancer norms. In balloon embodiments, cryoablative necrotizing energy is typically delivered to most or in the surrounding cavity wall, including both benign and malignant, by exposure to necrotizing cryoablative temperatures. Within the predefined treatment area, typically 5 mm to 15 mm, often, 5 mm to 20 mm, in depth of surrounding breast tissue and extending radially from the walls of the surgical cavity will undergo a necrotizing sub-freezing temperature gradient. Any remaining cells benign or malignant within this area will be necrotized, i.e. become non-viable.

An additional balloon configuration includes a secondary internal balloon to “pre-expand” the cryogenic cooling and tissue contacting balloon to effect increased system efficiency and reduce procedure time. Cryogenic fluid agents utilized are particularly useful for rapidly ablating (necrotizing soft tissue surrounding the walls of a surgical cavity). Suitable cryogenic fluids include, but are not limited to, carbon dioxide (CO₂), nitrous oxide, liquid nitrogen, near-critical nitrogen, perfluoropropane, propane, and other refrigerants such as R124, R1270, and R600a with unique phase change properties.

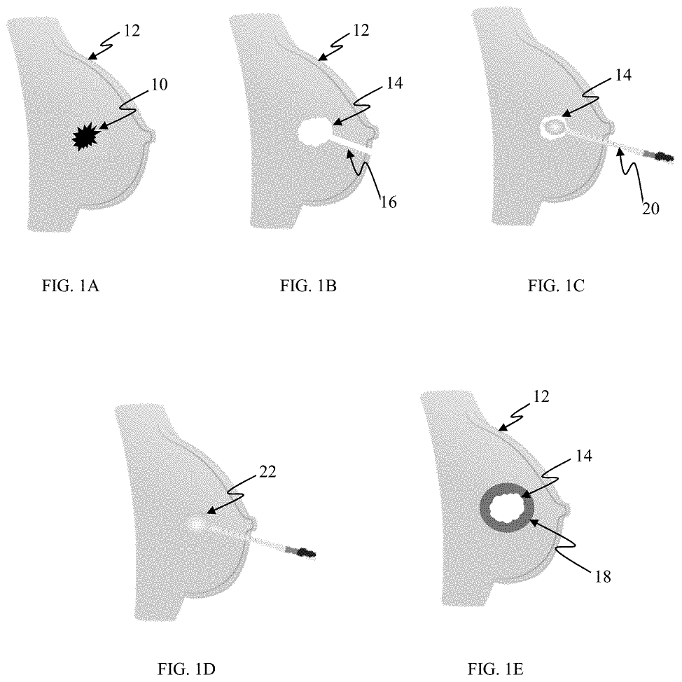

FIG. 1 illustrates in cross-section a breast at steps of treatment for the surgical excision of a breast cancer followed by balloon cryo-ablation of a margin of soft tissue in the walls of the surgical cavity. FIG. 1A illustrates a cancer 10 in the breast 12. FIG. 1B illustrates a surgical cavity 14 in breast 12 with a surgical tract 16 after the cancer has been excised. Surgical excision may be performed using open surgical techniques or using one or more devices that incorporate minimally invasive access. FIG. 1C illustrates balloon system 20 placed within the surgical tract with partial inflation of the balloon 22 in surgical cavity 14. FIG. 1D illustrates a fully expanded balloon 22 with cryogenic fluid and commencement of ablation of the margin of soft tissue in the walls of the surgical cavity. FIG. 1E illustrates the breast 12 after the balloon system has been removed and the surgical tract closed. A margin of soft tissue 18 within the wall of the surgical cavity 14 has been ablated.

To accommodate different cavity dimensions and shapes, the balloons can be provided in multiple different sizes and shapes typically including at least spherical and spheroid geometries. As the balloons will typically be non-distensible, as defined elsewhere herein, sizes can further be adjusted controlling inflation pressures. In double-wall balloon constructions, as described elsewhere herein, a space between inner and outer balloons can be maintained by introducing a cryofluid into a circulation region between the balloons at a higher pressure than an inflation pressure for the inner balloon.

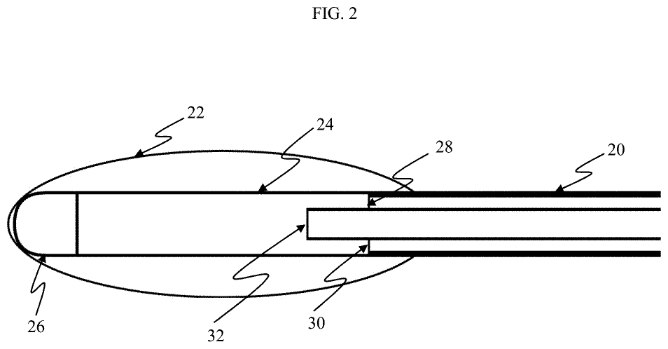

FIG. 2 illustrates a cross-sectional view of a single balloon cryo-ablation device 20. The tissue contacting balloon 22 is attached to support tube 24 and internally supported by end cap 26. Once the single balloon cryo-ablation device 20 is inserted into the breast, cryogenic fluid is infused into and circulated within the tissue contacting balloon 22 entering through supply tube 28 and, optionally a second supply tube 30, and exiting the tissue contacting balloon 22 through perforations within support tube 24 and a cryogenic fluid return tube 32.

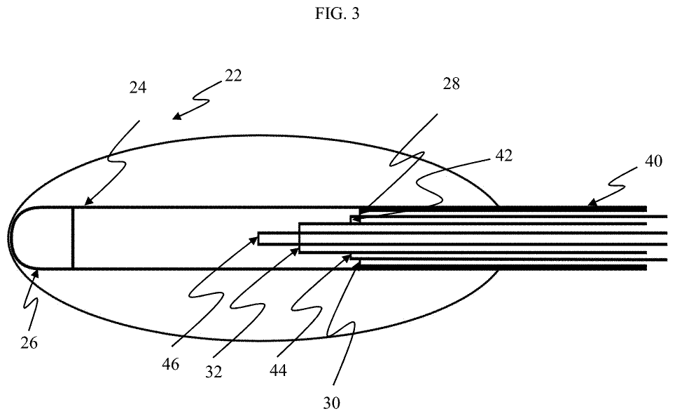

FIG. 3 illustrates a cross-sectional view of a single balloon cryo-ablation device 40 with the additional of a secondary fluid path. Once the single balloon cryo-ablation device 40 is inserted into the breast, a secondary fluid such as air, CO2 gas, saline, or other fluid media or gas is infused into and circulated within the tissue contacting balloon 22 entering from secondary fluid supply tube 42 and, optionally an additional secondary fluid supply tube 44, and exiting the tissue contacting balloon 22 through perforations within inner support tube 24 and secondary fluid return tube 46. Once the tissue contacting balloon 22 is expanded within the tumor cavity and drained of the secondary fluid, cryogenic fluid is infused into and circulated within the tissue contacting balloon 22 entering from supply tube 28 and, optionally additional supply tube 30, and exiting the tissue contacting balloon 22 through perforations within inner support tube 44 and cryogenic fluid return tube 32.

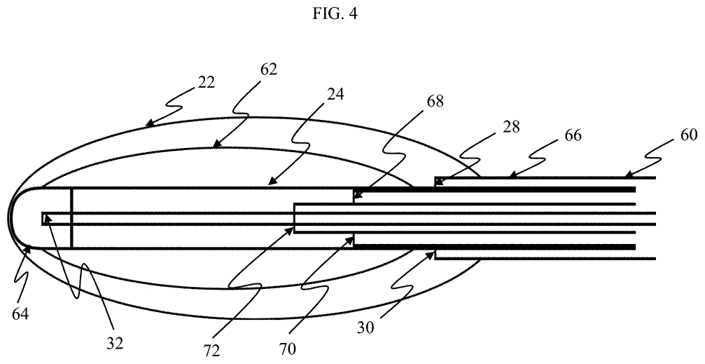

FIG. 4 illustrates a cross-sectional view of a dual balloon cryo-ablation device 60. The expansion balloon 62 is attached to inner support tube 24 and partially perforated end cap 64. A tissue contacting balloon 22 is attached to outer support tube 66 and internally supported by the partially perforated end cap 64. Once the dual balloon cryo-ablation device 60 is inserted into the breast, the expansion balloon 62 is infused with a secondary fluid and circulated within the expansion balloon 62 entering from secondary fluid supply tube 68 and, optionally an additional secondary fluid supply tube 70, and exiting the expansion balloon 62 through perforations within inner support tube 24 and secondary fluid return tube 72. Once the expansion balloon 62 is expanded within the tumor cavity, cryogenic fluid is infused into and circulated within the tissue contacting balloon 22 entering from supply tube 28 and, optionally additional supply tube 30, and exiting the tissue contacting balloon 22 through perforations within end cap 64 and cryogenic fluid return tube 32.

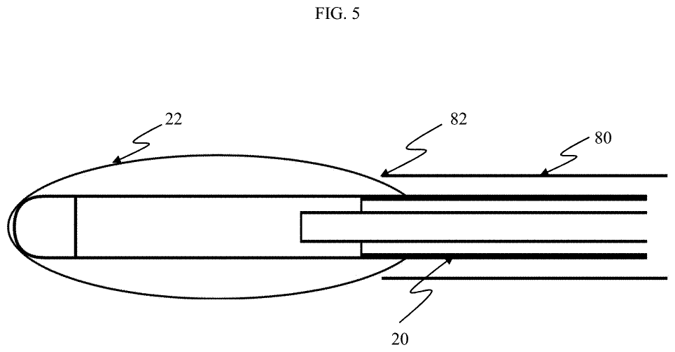

FIG. 5 illustrates a cross-sectional view of a single balloon cryo-ablation device 80 with the additional of a co-axial suction channel 82 around the periphery of the proximal portion of a single balloon cryo-ablation device 20 to remove excess air or fluid between the tissue contacting balloon 22 and the tissue cavity. Once this material has been removed, the cryo-ablation process can be initiated.

An exemplary protocol comprises:
Pre-cooled liquid cryofluid is pumped into the catheter connecting the cryo-balloon to the system console
Within the balloon the cryofluid warms by removing thermal energy from the surrounding tissue −6-8 minutes “freeze”
Within the console, the “used” (warmed) cryo-fluid is collected in a secondary container for subsequent reuse
At the end of the first freeze cycle the circulation path is reversed and the warmed fluid is used to thaw the balloon −8-10 minute “thaw”
A second 6-8 minute “freeze” is followed by a 8-10 minute “thaw”
The thawed and deflated balloon can then be easily removed from the surgical cavity

Any elements described herein as singular can be pluralized (i.e., anything described as “one” can be more than one). Any species element of a genus element can have the characteristics or elements of any other species element of that genus. The media delivered herein can be any of the fluids (e.g., liquid, gas, or combinations thereof) described herein. The patents and patent applications cited herein are all incorporated by reference herein in their entireties. Some elements may be absent from individual figures for reasons of illustrative clarity. The above-described configurations, elements or complete assemblies and methods and their elements for carrying out the disclosure, and variations of aspects of the disclosure can be combined and modified with each other in any combination. All devices, apparatuses, systems, and methods described herein can be used for medical (e.g., diagnostic, therapeutic or rehabilitative) or non-medical purposes.

What is claimed is:

1. A method for removing a tumor from solid tissue, said method comprising:
surgically removing the tumor to create a tissue cavity having an exposed tissue surface in the solid tissue;
inserting an expandable heat transfer surface surrounding an interior volume into the tissue cavity;
expanding the expandable surface against the exposed tissue surface of the body cavity; and
circulating a cooling fluid through the interior volume in order to cool and necrose the exposed tissue surface to a predetermined depth.

2. A method as in claim 1, wherein inserting the expandable heat transfer surface comprises inserting a single-walled balloon and expanding the balloon with the cooing fluid.

3. A method as in claim 1, wherein inserting the expandable heat transfer surface comprises inserting a double-walled balloon having an inner chamber and a space between an outer surface of the inner chamber and the expandable surface, wherein the cooling fluid is circulated through said space.

4. A method as in claim 3, wherein the inner chamber is expanded with a secondary fluid

5. A method as in claim 1, wherein the cooling fluid is pre-cooled.

6. A method as in claim 1, wherein the cooling fluid comprises a gas that undergoes a Joule Thomson expansion when introduced to the interior volume.

7. A method as in claim 1, wherein the cooling fluid comprises a liquid that has undergone an enthalpic expansion prior to circulation in the interior volume.

8. A method as in claim 1, wherein expanding the expandable surface comprises mechanically expanding the surface with a pull wire.

9. A method as in claim 1, further comprising stopping circulation of the cooling fluid through the interior volume of the expanded surface, circulating a warming fluid through the interior volume of the expanded surface, and circulating a cooling fluid the interior volume of the expanded surface a second time.

10. A probe for necrosing a marginal tissue region in a surgically created tissue cavity, said probe comprising:
a shaft;
an expandable heat transfer surface surrounding an interior volume at a distal end of the shaft, said expandable surface configured to conform to an inner surface of the surgically created tissue cavity when expanded therein; and
a cryogenic fluid supply lumen and a separate cryogenic removal lumen, both lumens being disposed in the shaft.

11. The probe of claim 10, wherein the cryogenic fluid supply lumen is disposed concentrically about the cryogenic removal lumen.

12. The probe of claim 10, further comprising a secondary fluid supply lumen in the shaft

13. The probe of claim 10, wherein the expandable heat transfer surface comprises a single-walled balloon.

14. The probe of claim 10, wherein the expandable heat transfer surface comprises a double-walled balloon having an outer balloon and an inner balloon, wherein the expandable heat transfer surface is on an outside of the outer balloon and both (a) a space between the outer surface of the inner balloon and an inner surface of the outer balloon and (b) an interior of the inner balloon are is configures to receive fluids.

15. The probe of claim 14, wherein the space between the outer surface of the inner balloon and an inner surface of the outer balloon is configured to receive the cryogenic fluid and the interior of the inner balloon is configured to receive a secondary fluid.

16. The probe of claim 10, further comprising a vacuum lumen configured to draw a negative pressure in a region surrounding the expandable heat transfer surface.
